(19) United States
(12) Patent Application Publication
(10) Pub. No.: US 2012/0089227 A1
(43) Pub. Date: Apr. 12, 2012

(54) HOLLOW HIGHLY-EXPANDABLE PROSTHETIC VERTEBRAL BODY

(76) Inventor: Peter Jarzem, Town of Mount Royal (CA)

(21) Appl. No.: 13/328,395

(22) Filed: Dec. 16, 2011

Related U.S. Application Data
(63) Continuation of application No. PCT/CA2010/000957, filed on Jun. 18, 2010.
(60) Provisional application No. 61/218,179, filed on Jun. 18, 2009.

(51) Int. Cl. A61F 2/44

(52) U.S. Cl. 623/17.12

(57) ABSTRACT

An expandable vertebral prosthesis for replacement of a vertebral body includes opposed end plates that are respectively adapted to abut two other vertebral bodies for fastening engagement therewith and a hollow expandable body interconnecting the end plates. The end plates each define a central opening which extends therethrough for receiving bone growth. The hollow expandable body includes one or more annular body sections having concentric inner and outer side walls, the inner side wall of which defines therewithin a central channel axially extending between the central opening defined in each of the first and second end plates. The central openings have a bone graft or bone growth stimulating material therein and the central channel permitting bone growth therethrough. The hollow expandable body is axially expandable such as to extend from a collapsed position to an expanded position whereby a space left by the excised vertebral body.

### CROSS-REFERENCE TO RELATED APPLICATIONS

The present application is a Continuation of International PCT Patent Application No. PCT/CA2010/000957 filed Jun. 18, 2010, which claims priority on U.S. provisional patent application No. 61/218,179 filed Jun. 18, 2009, the entire contents of both of which are incorporated herein by reference.

### TECHNICAL FIELD

The present invention relates generally to prosthetic vertebral bodies, and more particularly relates to an expandable prosthetic vertebral body.

### BACKGROUND

Vertebrectomy, the excision of a vertebra, is often employed to address several conditions which severely weaken the spinal vertebrae, in order to decompress the spinal cord and/or to stabilize the vertebral column, and thereby reducing the likelihood that a weakened vertebra may fracture and cause significant nerve injury. These conditions can include, but are certainly not limited to, cancer, infection, bone disease and genetic bone malformation, for example. Trauma or fractures can also necessitate such an excision of a vertebra.

Most known operative techniques for the excision of a vertebra, or a part thereof, are limited by the relatively restricted access to the vertebra which is to be removed and subsequently replaced and/or reconstructed. Most commonly, vertebrae are removed either from an anterior approach ((.e. via the front of a patient) or a posterior approach (i.e. via the back of the patient). Anterior approach techniques provide the widest access to the vertebra or vertebrae to be excised, however are sometimes associated with comorbidities with respect to the thoracotomy. Posterior approach techniques are generally preferred and are more frequently used as they are typically less morbid, however they imply considerable constraints in terms of limited access, as the vertebra must be excised and replaced with a suitable prosthetic replacement without damaging the nerve roots.

Prosthetic vertebral body “cages” have been used to replace a weakened vertebra, once removed. However, in order to fill the space created by the excised vertebra, such cages must typically be sufficiently large. Thus, most known vertebral body replacement cages are intended to be placed using an anterior approach, which allows for greater access. Such known cages cannot easily be positioned without causing unwanted damage, given the tight space constraints. The installation of such known vertebral cages via the patient's back (i.e. using a posterior approach) often requires resection of a nerve root in order to create a space large enough to permit cage entry. Existing cages therefore do not have sufficiently small size envelopes (whether diameter, length, etc.), or sufficient collapsibility, to readily permit entry thereof between nerve roots if installed using a posterior approach.

While some known vertebral cages can be used for both trauma and tumour indications, many existing cages tend to be better suited for tumour indications but less practical for trauma indications. Additionally, in cases where the removal of the vertebra is required, existing vertebral cages which permit some, but often not sufficient, expansion do not also lend themselves as well to efficient bone ingrowth.

Accordingly there remains a need for an improved prosthetic vertebral body.

### SUMMARY

In accordance with one aspect of the present invention, there is provided an expandable vertebral prosthesis, adapted for replacement of at least one vertebral body excised from between two other vertebral bodies, the vertebral prosthesis comprising: opposed end plates including outer surfaces thereon which face in opposite directions and are respectively adapted to abut said two other vertebral bodies for fastening engagement therewith, the end plates defining a central opening which extends therethrough; a hollow expandable body interconnecting the end plates by one or more annular body section having radially spaced apart inner and outer side walls, the inner side wall defining therewithin a central channel axially extending between the central opening defined in each of the first and second end plates, he central openings permitting bone ingrowth therein and the central channel permitting bone growth fully therethrough between the two opposed end plates; and wherein the hollow expandable body is axially expandable such as to extend from a collapsed position to an expanded position, the expanded position filling a space left by the at least one excised vertebral body.

There is also provided, in accordance with another aspect of the present invention, an expandable vertebral prosthesis comprising opposed first and second end plates spaced apart by a hollow expandable body and including outer surfaces thereon which are respectively adapted to abut adjacent vertebral bodies for engagement therewith, the end plates each having a bone ingrowth opening extending therethrough, the hollow expandable body having an annular configuration defining a central channel longitudinally extending therethrough between the bone ingrowth openings in the end plates, the central channel permitting bone growth fully therethrough between the opposed first and second end plates, the hollow expandable body having an external side wall and an internal side wall enclosing a sealed annular cavity therebetween, the internal side wall enclosing the central channel extending between said openings in the end plates, the annular cavity defined within the body being adapted to receive and contain a hardenable material therein, at least one inlet port being provided in fluid flow communication with said annular cavity to permit injection of the hardenable material into said cavity thereby expanding the body to expand the vertebral prosthesis in a longitudinal axial direction such that the end plates are displaced away from each other, the vertebral prosthesis being axially expandable when the annular cavity is filled with the hardenable fluid to extend the vertebral prosthesis from a collapsed position to an expanded position in order to fill a space between said adjacent vertebral bodies.

There is further provided, in accordance with another aspect of the present invention, an expandable vertebral prosthesis comprising opposed first and second end plates spaced apart by a hollow expandable body and including outer surfaces thereon which are respectively adapted to abut adjacent vertebral bodies for engagement therewith, the end plates each having a bone ingrowth opening extending therethrough, the hollow expandable body having an annular configuration defining a central channel longitudinally extending therethrough between the bone ingrowth openings in the end plates, the hollow expandable body having an external side wall enclosing the central channel extending between said openings in the end plates, the hollow expandable body including three or more concentric barrels nested together and relatively displaceable such as to form a telescopic central body of the vertebral prosthesis, the telescopic central body being extendable to expand the vertebral prosthesis in a longitudinal axial direction such that the end plates are displaced away from each other, the telescopic central body extending a determined distance to expand the vertebral prosthesis from a collapsed position to an expanded position in order to fill a space between said adjacent vertebral bodies.

### BRIEF DESCRIPTION OF THE DRAWINGS

Further features and advantages will become apparent from the following detailed description, taken in combination with the appended drawings, in which:

FIG. 1 is a perspective view of a prosthetic vertebral body in accordance with one aspect of the present application, shown in an expanded position between two vertebrae;

FIG. 2 is a perspective view of the prosthetic vertebral body of FIG. 1, shown in an expanded position;

FIG. 3 is a perspective view of the prosthetic vertebral body of FIG. 1, shown in a collapsed position;

FIG. 4 is a top plan view of the prosthetic vertebral body of FIG. 1;

FIG. 5A is a perspective view of the prosthetic vertebral body of FIG. 1;

FIG. 5B is an enlarged detailed view of portion 5B of the side wall of the prosthetic vertebral body of FIG. 5A;

FIG. 6 is a perspective view of a prosthetic vertebral body in accordance with an alternate aspect of the present disclosure, shown in an expanded position; and

FIG. 7 is a perspective view of a prosthetic vertebral body in accordance with another aspect of the present application, shown in an expanded position;

FIG. 8 is a perspective view of a prosthetic vertebral body in accordance with another aspect of the present application, shown in an expanded position; and

FIG. 9 is a perspective view of a prosthetic vertebral body in accordance with another aspect of the present application, shown in an expanded position.

### DETAILED DESCRIPTION

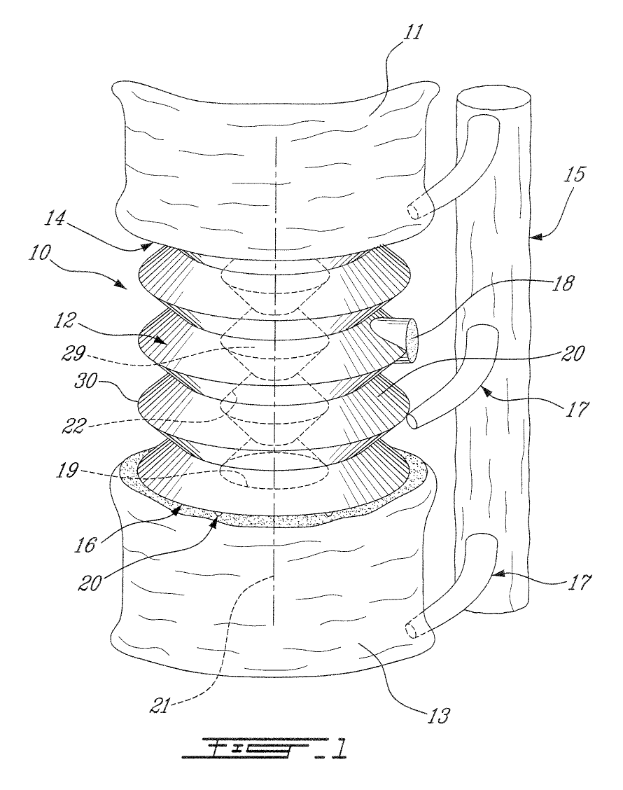

Referring to FIG. 1, a vertebral prosthesis 10 in accordance with one embodiment of the present invention is shown in an expanded position, installed in place between two vertebrae 11 and 13. The spinal cord 15 is shown schematically and includes nerve roots 17 for each vertebra. The vertebral prosthesis 10 is thus used to replace one or more excised vertebra, a portion of a vertebra and/or to stabilize and fix the spinal column of a patient, especially in conjunction with a spinal resection in which the vertebral prosthesis or implant 10 is braced between upper and lower vertebrae, such as vertebrae 11 and 13 shown in FIG. 1. Vertebral implants are sometimes referred to as “cages”, because they have traditionally consisted of metallic cage-like structures. The present vertebral prosthesis 10 may be either used alone to stabilize and fix the spinal column by replacing an excised vertebral body, or alternately may be used in combination with supplemental fixation (not shown), either posterior or anterior, in order to augment the cage fixation. Thus supplemental fixation can include rod and pedicle type screw systems. Typically, if the prosthesis is placed using a posterior approach, the supplemental fixation is also disposed posteriorly. The opposite would be true if an anterior approach is used.

As seen in FIGS. 1-3, the present vertebral prosthesis (VP) 10 includes opposed first and second end plates 14 and 16 that are interconnected by a generally tubular body 12. The end plates 14,16 each define an opening 19 therein, which opening extends fully transversely through the end plates, such as to define generally annular end plates 14,16. The openings 19 in each of the end plates may be centrally located within perimeters of the circular end plates as shown, or alternately may be offset, particularly in cases where the end plates 14,16 are not circular. For example the end plates, may have D-shaped, kidney-shaped, oval, rectangular, square or other shaped perimeter profiles for example.

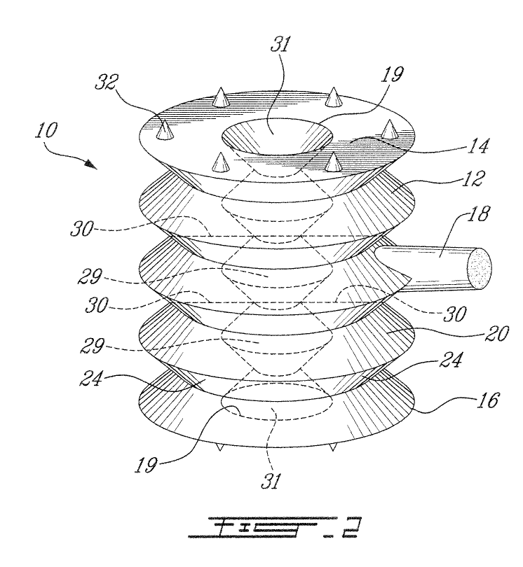

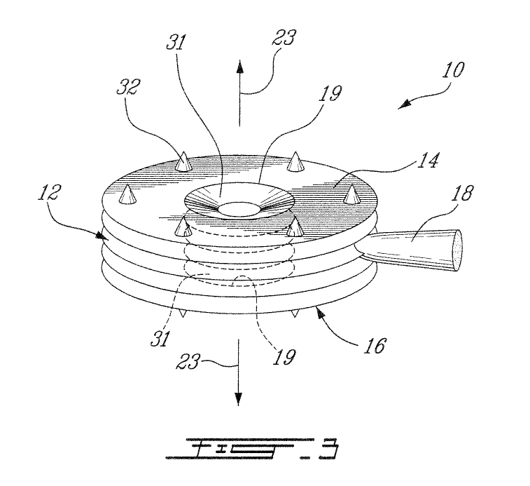

A central channel 29 extends through the length of the VP 10 between each of the openings 19 in the end plates. Thus, the VP 10 is said to be “hollow” as the term is defined herein, i.e. the VP includes a longitudinally extending passage or channel therethrough that is open at either end of the device, extending between the openings 19. The tubular body 12 of the device 10 is therefore substantially annularly shaped, having an external side wall 20 and an internal side wall 22 which together with the end plates 14,16, for an enclosed annularly-shaped cavity 24 which is adapted to receive a hardenable material, such as a polymerizing fluid, which is injected into the cavity 24 via the filler port 18 such as to force the expansion of the VP 10 from a collapsed position, as shown in FIG. 3, to an expanded position, such as shown in FIG. 2. This annular configuration of the body 12 of the VP 10 provides for excellent strength, and very little if any loss in strength occurs as a result of the “hollow” central channel. The VP 10 may in fact be able to support greater loads than comparable “solid” VP structure (i.e. without a central channel 29 extending therethrough). Further, the annular body 12 of the VP 10 corresponds substantially to the vertebral bodies 11,13 between which the VP 10 is disposed, given that vertebrae have stronger bone material around their perimeter and softer bone near their centers.

It is of note that although the end plates 14,16 and the body 12 of the VP are depicted as being substantially circular in shape and peripheral profile, this need not necessarily be the case. Further the internal side wall 22 and the external side wall 20 need not be the same shape.

Although the internal side wall 22 and the external side wall 20 may be substantially parallel to each other, as is the case of the accordion-like sidewalls shown in FIGS. 1 and 2 for example, they need not necessarily be identical in shape or form. In one preferred, but not essential, embodiment, the internal and external side walls 22.20 substantially mirror each other such as to improve the structural integrity of the VP. However, both the internal and external side walls must be able to substantially equally expand such as to permit the VP 10 to be displaced from the very compact collapsed position (FIG. 3) to the significantly expanded position (FIG. 2), as required. Although the end plates 14,16 need not remain exactly parallel to each other (i.e. exactly perpendicular to the central longitudinal axis 21), neither the internal or external side wall 22, 20 must restrict the expansion of the VP 10 as the annular cavity 24 is filled with the hardenable material.

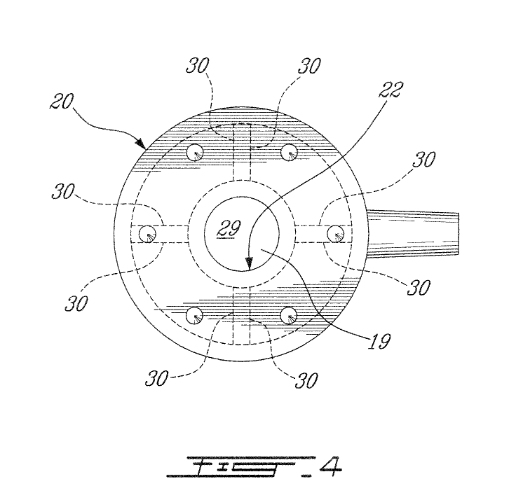

The longitudinally extending channel 29 connecting the openings 19 in the end plates 14,16 is preferably maintained substantially centrally within body 12 of the VP 10, in order to maintain desired structural integrity and prevent one side of the VP 10 to be weaker than another. In order to help locate and maintain the channel 29 centrally through the body 12 of the VP 10 as it expands, a number of “stringers” or stabilizing ties 30 are provided within the annular cavity 24 and which extends substantially radially such as to link the internal side wall 22 with the external side wall 20. As best seen in FIG. 4, these stringers 30 may be provided in pairs and disposed at substantially 90 degree intervals about the periphery of the internal side wall 22, in order to provide four pairs of stringers 30 extended between the internal and external side walls 22, 20. As seen in FIG. 2, two separate such groups may be provided, each at a different vertical position within the cavity 24, in order to securely position the longitudinally extending cavity 29 substantially centrally within the body 12 of the device, regardless of how the body needs to expand to fit between next adjacent vertebrae 11,13 when filled with the hardenable material.

The openings 19 in the end plates 14,16 are adapted to accept and receive a bone graft or bone growth stimulating material 31 therein, prior to installation of the VP 10, such that the bone growth stimulating material is able to grow through the central channel 29 enclosed by the internal side wall 22 of the VP body 12, in order to be able to eventually link or fuse the two vertebrae 11 and 13 together. When in the body 12 of the VP 10 is in the collapsed position, the bone growth stimulating material may fill the central channel 29 extending between the openings 19 in the end plates, given that the end plates are very closed together in this collapsed position of the device. The bone growth stimulating may be, for example only, a paste or a powder mixed with blood which is packed within the openings 19 or alternately may be provided on a collage sponge disposed within the openings 19.

The central channel 29 extending through the body 12 of the VP 10 therefore allows for fusion between vertebrae through the device. Thus, the VP 10 can be used for trauma patients when bone growth stimulating is provided within the openings 19 for ingrowth through the channel 29. Accordingly, this makes the VP 10 better suited for passing 510k certification by the FDA.

In an alternate embodiment, the hardenable material that is injected into the annular cavity 24 of the body 12 may be an antibiotic eluting cement, which allows for the use of the device 10 in patients who have infections.

The first and second end plates 14 and 16 define outer surfaces respectively, which form the two outwardly facing surfaces of the VP 10 that are adapted to abut the two adjacent vertebrae 11 and 13. As described further below, the end plates 14.16 are preferably fastened or anchored to the adjacent vertebrae 11,13 using surface features formed on their outer surfaces. These surface features may include, for example, projections 32 which help to anchor the VP in place between the two next adjacent vertebral bone structures. In one embodiment, these surface features include a plurality of textured protrusions 32 which extend from the outer surface of each of the end plates 14, 16, such as to permit the end plates to anchor and/or fasten to the bone structures surrounding the VP. The protrusions 32 can include: teeth, pins, barbs, spikes, and any combination thereof. The surface features can also include non-protruding surface feature elements, either in addition to or in place of the protrusions, which nonetheless help the end plates to be engaged, anchored and/or become fastened to the bone structure of the surrounding vertebrae. These non-protruding elements 27 can include, for example, porous ingrowth surface regions, bioactive bone growth materials, and at least one opening for receiving a bone screw, whereby the end plate is screwed directly in place on the vertebra.

The tubular body 12 of the VP device 10 has an expanding configuration which allows at least for expansion of the body of the VP 10 such as to fill any sized opening between vertebrae. Particularly, the body of the VP generally may expand along a longitudinal axis 21 of the VP, however it is to be understood that deviations from the axis are of course possible. Regardless, the VP 10 expands such that the end plates 14,16 are generally displaced away from each other. However, the two end plates 14,16 need not remain parallel to each other, and therefore the body can expand to accommodate any slope of the endplates 14,16 necessary for their outer surfaces to abut the adjacent vertebrae 11,13, even a slope that is significantly canted from a plane which is perpendicular to the longitudinal axis 21 of the cage.

The filler inlet port 18 communicated with the annular cavity 24, and therefore the hardenable material is injected through this inlet filling port 18 into the annular cavity 24. This hardenable material filling port 18 may be disposed, for example, in the external side wall 20 as shown in FIGS. 1-3, or alternately proximate one of the two end plates (not shown). Other positions of the filler inlet port 18 are of course also possible, provided that the inlet port is disposed in fluid flow communication with the internal annular cavity 24 defined within the VP 10. Preferably, the polymerizing fluid which is used is a bone cement paste, that hardens once the VP has been forced into the expanded position sufficient to fill the space left by the excised vertebral body or bodies that the VP 10 is replacing.

In addition to the filling port 18 through which the hardenable material is injected, the VP 10 may also be provided with a distinct, second filling port (not shown) which communicates with the central channel 29 within the body 12 of the VP 10 and may therefore be used to inject bone growth stimulating material (such as bmp, bone slurry bmp, etc.) into this central channel 29 of the VP 10, whether disposed in the collapsed or expanded position thereof.

The combined axial height (i.e. thickness in a direction substantially parallel to the longitudinal axis 21) of the two end plates 14,16 is relatively small compared to the total axial length (i.e. height) of the VP. This enables the VP 10 to be compressed into much smaller space envelopes than the devices of the prior art, thus enabling the placement of VP 10 via much smaller surgical access openings, and in particularly enabling the placement of the VP 10 via a posterior approach without causing undue damage to the surrounding nerve and tissue structures. The VP 10 thus provides an implant which can be inserted through a relatively small insertion opening, such as through a small posterior surgical access, between pairs of nerve roots, through a costotransversectomy or a wide transpedicular approach, for example. The VP 10 thus has a collapsed position shown in FIG. 3, which defines a small size envelope for ease of insertion, but which can subsequently be expanded to fill a much larger space, as shown in FIGS. 1-2 for example. This is achieved as the VP 10 has a body 12 which has an expanding configuration allowing for expansion of the VP. The body 12 has side walls 20,22 that may have a variety of different configurations, as described further below, however regardless of configuration, the side wall 20,22 of the body 12 are such that the end plates 14,16 of the VP 10 are displaced away from each other, when the cavity is expanded by the injection thereof of bone cement, such that the VP expands to fill a given opening between vertebral bodies.

The VP 10 includes first and second end “plates” 14 and 16 which are interconnected by the generally tubular side wall 12, such as to define a cavity within the VP. Although the term “plates” is used to define the end surfaces of the body which makes up the VP, it is to be understood that these plates may be integrally formed with the material of the side wall 12, and may also not necessarily be smooth or flat. The end plates 14 and 16 may also be disposed either externally or internally within an outer sheath or casing made up by the material of the side walls 20,22 of the body 12 which extends over the plates 14,16 at either end. Thus, the plates can constitute a thin walled material, such metal or a polymer (such as a bioresorbable polymer for example), which is either integral with, or separate and fastened to, the material of the side walls. The end plates 14, 16 are however preferably, but not absolutely, harder and/or stiffer than the side walls 20,22 of the body 12, whether the end plates are made of a different material or not.

As noted above, both the external side wall 20 and the internal side wall 22 of the VP body 12 has a configuration which permits expansion of the VP 10 generally in the opposed directions 23, as shown in FIG. 3, which may in one embodiment be substantially parallel to the longitudinal axis 21 of the VP. Various configurations of side wall 12 are possible to achieve such an expansion, however in the embodiment depicted in FIGS. 1-5B, the side wall 12 has a plurality of accordion type pleats which give the side wall an expanding bellows type folded shape. This folded, tubular side wall configuration thus enables the end plates 14 and 16 to be displaced towards and/or away from each other in a generally longitudinal direction. The accordion pleats 30 of the side wall 12 will prevent the device from unduly expanding in a radial direction and restricts most expansion to the opposed longitudinal directions 23, thus protecting the spinal cord from inadvertent injury when the VP is placed in position between vertebrae and expanded. Further, the flexibility provided by such a wall design permits the two end plates 14 and 16 to be angled, or canted, as required in order to accommodate the specific local topography of the vertebrae against which they are abutted when the VP 10 is expanded in situ. Thus, the end plates 14,16 are free to be disposed, when the VP is expanded in place between the two adjacent vertebrae 11,13, at different angles relative to the longitudinal axis 21 (i.e. the two end plates need not be parallel to each other).

The accordion pleat structure of the side wall 12 permits this cant angle mismatch between the two opposed endplates without significant radial displacement of the side walls of the device. In other words, the end plates 14,16 of the VP 10 can automatically (that is, by themselves without requiring outside aid) adjust their angulation to the specific angles of the bone structures to which they are to be attached, as the internal cavity of the VP is filled with the bone cement that forces the two end plates apart from each other and into contact with their adjacent vertebrae. Further, as the VP expands, the bellows structure of the side walls permits the two end plates to be offset from each (in addition to being at different angles) if necessary, i.e. their center points are not axially aligned with each other or with the central longitudinal axis 21.

The side walls of the body of the VP 10 may be made of any material that is thin walled and flexible, and suitable for biological applications. These can include metal, plastic or polymer, such as a resorbable polymer for example. The end plates may be made of the same material as the side walls, or alternately of a different material, such as a more rigid metal, plastic or composite for example.

Other expanding wall configurations are possible, in addition to the accordion type design described above.

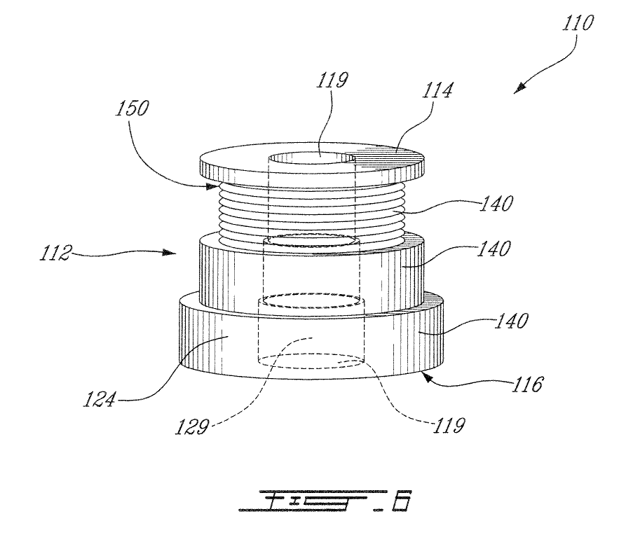

For example, the alternative VP 110 as shown in FIG. 6, which has a telescoping, multi-tier (or “wedding cake”) design. With such a telescoping design, a number of segments, tiers or layers 140 fit within each other when the device is in the collapsed position, and these wall segments telescope with respect to each other and mechanically interlock, for example by an anti-collapse mechanism, without necessarily requiring any central filling fluid for structural support or to expand the VP 110 into an expanded position as shown in FIG. 6. However, the VP 110 having such a telescoping, multiple tier design nonetheless includes a central passage 129 which extends longitudinally through the device between openings 119 at each end thereof, namely in each of the two opposed end plates 114 and 116, within which may be received a bone growth stimulating material and such that bone in/through-growth can occur. The VP 110 thus includes a plurality of tubular barrels 140 which are concentric and have different diameters such as to be nested together and thus displaceable from their collapsed position to an extended position, where the barrels 140 are displaced relative to each other to form the multiple-tier wedding cake like expanded position of the VP 110, as shown in FIG. 6. Preferably, three or more of the tubular barrels 140 are provided, such as to form a multiple-tiered expandable telescoping body of the VP.

The VP 110 of FIG. 6 therefore includes the opposed end plates 114, 116 which are separated by an extendable and/or expandable central body 112. The end plates 114, 116 may each be provided with an opening 119 therein which allow for and/or promote bone ingrowth into at least the end plates (and may, for example, have bone growth stimulating material provided therein prior to installation of the VP), and more particularly the entire body of the VP. For example, the central body 112 of the VP 110 may be generally annular or otherwise may be formed having a central passage 129 extending therethrough and which interconnects the two openings 119 at either end of the device. Thus the VP 110 may define a longitudinally extending passage 129 such that the device is fully hollow through its center, to allow for bone through growth.

In the particular embodiments of the expandable, multiple tier, vertebral prosthesis 110, 210, 310 and 410 as shown in FIGS. 6-9, the VP of the present application may be a so-called “all-metal” cage, in that it does not require or employ bone cement to expand the device nor to render it rigid and fix it in place between vertebrae 11, 13, as will be described in further detail below. It is however to be understood that these VPscan be made of materials other than metal, for example plastic, ceramic, composites and/or other biocompatible materials.

In all of these embodiments, the telescoping configuration of the central body 112, 212, 312, 412 of the VPs 110, 210, 310, 410 are made up of a number of substantially annular barrels 140, and more preferably three or more of such annular barrels 140 are provided. The central bodies 112, 212, 312, 412 of the VPs 110, 210, 310, 410 all also comprise an expansion mechanism 150 that interconnects and/or interlocks the barrels 140 such as to permit controlled expansion of the central body and thus expansion of the entire VP within the gap left between the vertebrae 11, 13, without requiring the use of bone cement to do so. Particularly, the expansion mechanism 150 defined within the barrels of the VP 110 may comprise one or more of a number of different possible mechanisms which are actuable to displace the nested barrels, and thus the opposed end plates 114, 116, at least away from each other a determined and controlled distance.

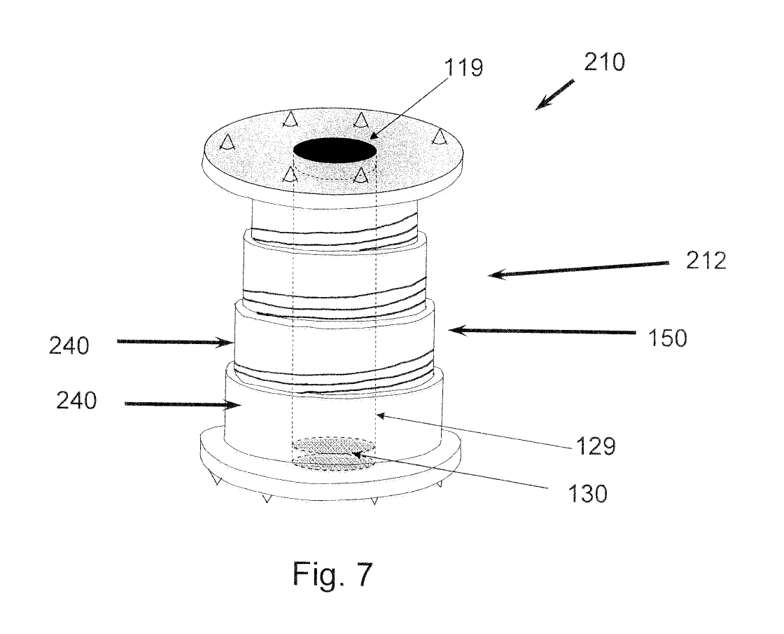

For example, the expansion mechanism 150 may include an interlocking screw design as depicted in FIG. 6. One or more of the barrels 140 may thus have threads on an outer surface thereof (as depicted on the uppermost barrel 140 in FIG. 6), which fit within corresponding threads within the next adjacent barrel 140. It is to be understood that all of the barrels 140 may be similar threaded, despite the fact that only one is shown with threads in FIG. 6 for simplicity. In the alternate embodiment of FIG. 7, the VP 210 includes a greater number of barrels 240, wherein four (or more) barrels 240 are all interconnected for telescopic expansion by an expansion mechanism 150. The expansion mechanism 150 of VP 210 also includes mating threaded engagement between each successive barrel 240 (schematically illustrated as an example only).

In this screw design, such as per the expansion mechanism 150 provided in both VP 110 and 210 of FIGS. 6 and 7, one or more of the barrels 140,240 are threadably connected to the next adjacent tier-defining barrel 140,240 and/or to the end plates 114. 116, such that rotation of at least one of the barrels will cause the entire VP 110,210 to longitudinally expand.

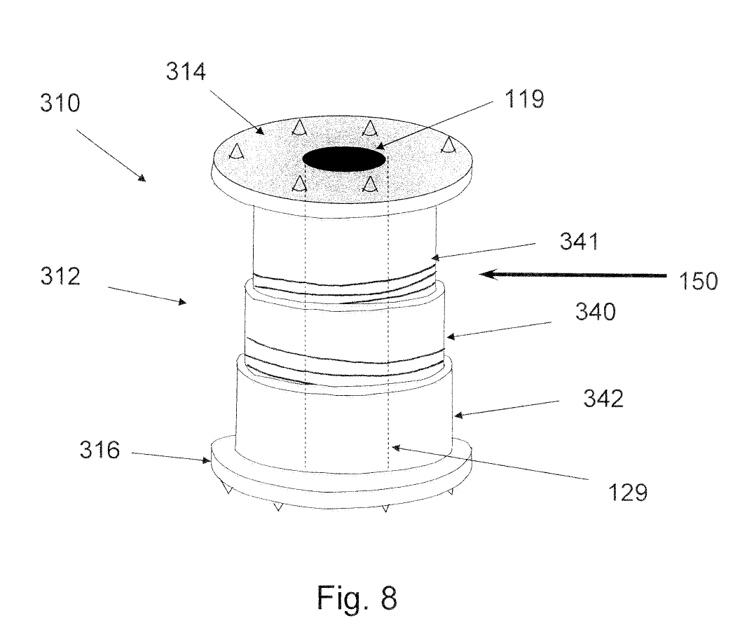

In the embodiment of FIG. 8, the VP 310 differs from those described above in that the expansion mechanism 150 alternately includes a turnbuckle design. In this three-piece turnbuckle expansion mechanism design, the upper barrel 341 screws counter-clockwise into the center or second barrel 340, which itself screws clockwise into the lower or third barrel 342. In the illustrated embodiment, the center and upper barrels 340, 341 are externally threaded, while the lower barrel 342 is internally threaded such as to threadably receive the central barrel therein. In an alternative embodiment, the upper barrel 341 is larger than the center barrel 340 and is internally threaded such as to threadably receive the central barrel therein. The two end plates 314. 316 remain rotationally fixed with respect to their respective adjacent barrels 341, 342. Therefore, with this turnbuckle design of the expansion mechanism 150, by rotating the central barrel 340 clockwise, the upper and lower barrels 341,342 are displaced outward with respect to the central barrel (i.e. away from each other along the longitudinal axis of the VP). Although only three barrels are shown in FIG. 8, a five barrel VP having such a turnbuckle-type expansion mechanism 150 may also be used, whereby, for example, an additionally externally threaded barrel (a fourth barrel) similar to barrel 340 is added below the third barrel 342, and another added barrel (a fifth barrel) similar to the upper barrel 341 is added below the new fourth barrel. In these designs, rotation of the central barrel causes the two end plates to be displaced away from each other, or towards each other if the direction of rotation of the central potion is reversed.

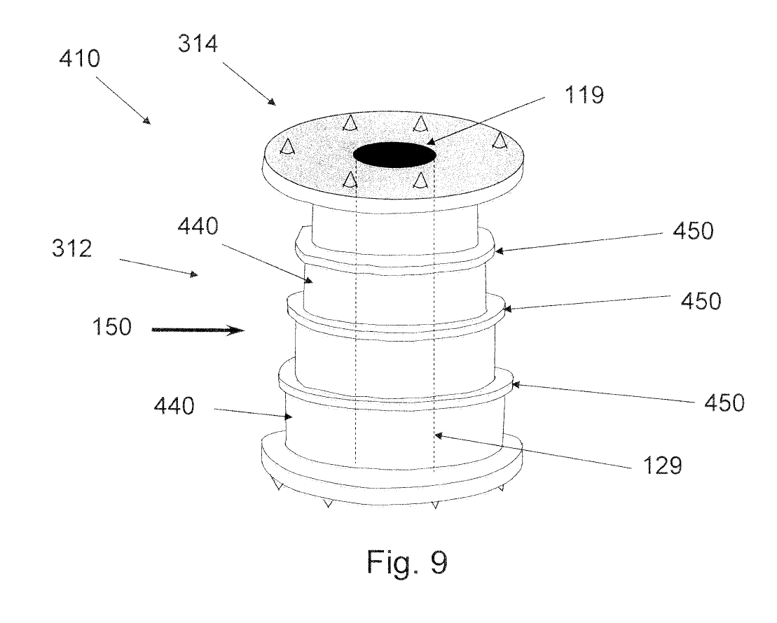

In the embodiment of FIG. 9, the VP 410 includes four barrels 440 all of which have ratchet teeth formed in the external walls thereof (not visible in FIG. 9) and the expansion mechanism 150 includes a ratchet like design, wherein a ratchet ring 450 is disposed on each of the barrels 440. Rotation of one or more of the ratchet rings 450 will thus cause the respective barrels to be displaced outward relative to each other in order to thereby expand the VP 410 longitudinally.

It is to be understood that other embodiments of the expansion mechanism 150 may also be used, for example a scissor jack type design which interconnects the barrels for expansion of the VP when the jack is extended.

It is of note that in all of the VP embodiments depicted in FIGS. 6-9, each end plate still includes the opening 119 therein for receiving bone growth stimulating material 130 (see FIG. 7 for example) therein, as well as the longitudinally extending central channel or passage 129 extending between the two openings in the opposed end plates.

Thus, relative expansion of the concentric and interlocking barrels 140, which thereby provide multiple concentric and stacked (i.e. nested) expansion cylinders, allows for expansion ratios much greater than with a single telescope design. Particularly, where a single-tier telescope design would allow for approximately a 60-70% expansion ratio, the present multiple-tier expandable body 112 of the VP 110 allows each of the three or more barrels, or tiers, of the body 112 to open 60%. Although more than two barrels or tiers 140 are preferably provided, for example the three barrel configuration shown in FIG. 6, it is to be understood that the expandable, telescoping body 112 of the VP may have, three, four, five or more of such barrels/tiers. For example, in one embodiment the body 112 has four such barrels 140, would provide collectively a 240-280% expansion (i.e. 4×60-70%). In another embodiment, the VP 110 includes five concentric barrels 140 which are interconnected and expandable such as to provide an expansion of 300-350% (5×60-70%) for the VP. In either case, such an expansion ration (i.e. between 240 and 350%) allows for a highly expandable VP cage which is at least sufficient to replace one vertebra.

In all cases, the expansion mechanism 150 can be adjusted a controlled amount such as to expand the overall height of the VP 110, as required given the particular anatomical requirements. Additionally, the expansion mechanism 150 permits such controlled displacement without requiring any bone cement to be introduced into and/or around the VP 110. Thus, there is no cement injection and the barrels 140 which make up the tiers are held in their expanded position (ex: as shown in FIG. 6) by the expansion mechanism 150 which is integrated directly into the barrels 140 themselves.

Other expanding body configurations are of course also possible, such as ones having a diamond shaped, braided and/or spiral geometric side wall structure. A Chinese finger trap type orientation of the fibres of the side wall can also be used. Regardless of the particular design, two substantially parallel side walls (namely one internal and one external) define both the central longitudinally extending cavity 29, 129 extending through the VP and the annular, enclosed cavity 24, 124 within which bone cement or another hardenable material is injected such as to cause the expansion of the device and maintain its structural integrity once expanded.

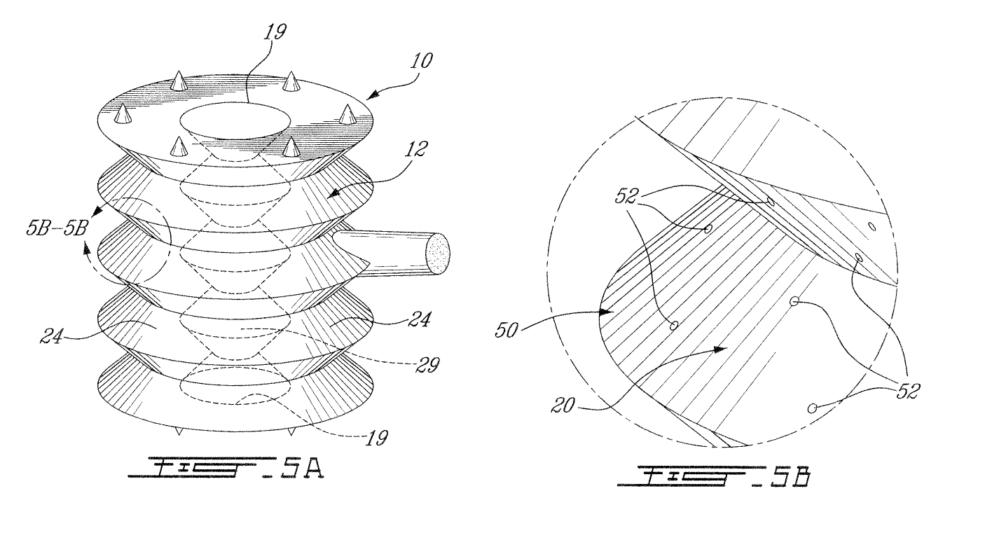

Referring now back to FIGS. 5A-5B, the VP 10 preferably includes an integrated air evacuation system. The VP 10 may have an integrated micropore air evacuation system 50, comprising a plurality of microscopic, or at least very small, air evacuation holes 52 defined through at least the outer or external side wall 22 of the body 12. These air evacuation holes 52 permit the evacuation of air from the internal annular cavity 24 within the VP 10, such as to prevent air entrainment. The holes 52 are however sufficiently small in size to prevent the relatively more viscous polymerizing fluid (ex: cement) from escaping from the internal cavity through the walls of the VP 10, as this polymerizing fluid is significantly more viscous than air. Such micro-pores 52 may also be provided in the internal side wall 22.

In use, the VP 10 collapses into a very small size envelope, such as to make its insertion into place between the nerve roots of two adjacent vertebrae possible without causing damage, even upon a posterior placement. Although the distance between adjacent nerve roots varies along the spine, this distance is generally between about 1 cm and about 2 cm. Accordingly, when the VP 10 is disposed in its fully collapsed position, it has a total collapsed height of less that about 1-2 cm. As the end plates 14,16 are very thin relative to the total potential height of the entire device 10, the fully collapsed position (FIG. 3) can be much smaller than most existing cage designs of the prior art, particularly relative to their expansion potential. For example, in one embodiment, a combined axial height of the end plates 14,16 is at most 20 percent of the total height of the VP 10 when it is disposed in the fully collapsed position, as shown in FIG. 3 for example. Thus, in this embodiment, the tubular side walls 20,22 of the VP body 12 thereby have an axial height of at least 80 percent of the total height of the VP, in the fully collapsed position. In another more specific embodiment, the combined axial height of the end plates makes up at most 10 percent of the total height of the device, when the VP 10 is disposed in the fully collapsed position. Thus, in this embodiment, the tubular side wall 12 thereby has an axial height of at least 90 percent of the total height of the VP when disposed in its most collapsed position. It is to be understood that the term “total height” as used herein is intended to mean the longitudinal axial distance between the outer surfaces of the first and second end plates 14,16 of the VP 10. Thus, for a similarly sized fully expanded height, the fully collapsed height of the VP 10 is much small than those of the prior art.

Thus, one feature of the VP 10 is that it can be greatly collapsed, permitting significantly higher expansion ratios (i.e. the total expanded height divided by the collapsed height), such as, in one particular embodiment, expansion ratios ranging from about 200% to about 850%. However, it is to be understood that the expansion ration can vary anywhere from as small as 20% to as large as 2000%. in one particular embodiment of the VP 10, however, this expansion ratio is at least 200%. In another embodiment, the expansion ratio is greater than 300%. In yet another specific embodiment, the expansion ratio is up to about 850%, which corresponds for example to the expansion ratio needed for a three level cage in a large person in the lumbar spine. The VP 10 is therefore able to fill defects of about 60 mm, 45 mm and 22mm in size for single level vertebral bodies in the lumbar, thoracic and cervical spines respectively. For two and three levels (i.e. where two and three vertebrae are being replaced), the size defects which the VP 10 is capable of filling would correspond to two and three times these values, respectively.

This is at least partly possible due to the relatively thin end plates. In one embodiment, the combined axial height of the first and second end plates is less than 5% of the total height of the entire VP 10 when it is disposed in the expanded position. This compares to prior art designs, in which at least 65% of the overall height of the entire cage is taken up by the thick endplates. For such prior art designs, expansion ratios are also much smaller, such as of the order of about 140%

In one possible example, these end plates 14,16 are about 3 mm thick each and the height of the collapsed bellows side walls 20,22 of the body 12 is about 54 mm, for a total collapsed height of 60 mm for the VP 10. Given that the bellows-like body 12 of the device can expand about 300% (i.e. 54 mm×3=162 mm), then the VP 10 would be able to expand to a total height of about 168 mm (i.e. 3 mm+162 mm+3 mm). This corresponds to an expansion ratio of about 280%. Thus, for a given total collapsed height, the present VP 10 is capable of expanding to fill a much bigger defect gap than is possible with any device of the prior art. As such, the VP 10 is capable of being expanded to fill a gap left by 1, 2 or 3 excised vertebrae, for example.

If viewed in an alternate manner, given a fairly typical 70 mm vertebral defect height which can exist after a thoracolumbar or lumbar vertebrectomy for example, the VP 10 of the present invention could be inserted therein having a 21.3 mm minimum (i.e. collapsed) height, which enables its insertion between pairs of nerve roots from a posterior approach, prior to being sufficiently expanded to adequately fill the 70 mm defect opening. In another embodiment of the present invention, the VP 10 has an overall expansion ratio of 400-500%, which results in an minimum entry height of the collapsed device about 14 mm possible for a 70 mm space. This is clearly a large improvement over the devices of the prior art, wherein the expandable cages would typically have a minimum (collapsed) height of 60 mm, which is far too large to be inserted from the posterior approach.

The embodiments of the invention described above are intended to be exemplary. Those skilled in the art will therefore appreciate that the forgoing description is illustrative only, and that various alternatives and modifications can be devised without departing from the spirit of the present invention. Accordingly, the present is intended to embrace all such alternatives, modifications and variances which fall within the scope of the appended claims.

1. An expandable vertebral prosthesis, adapted for replacement of at least one vertebral body excised from between two other vertebral bodies, the expandable vertebral prosthesis comprising:
opposed end plates including outer surfaces thereon which face in opposite directions and are respectively adapted to abut said two other vertebral bodies for fastening engagement therewith, the end plates defining a central opening which extends therethrough;
a hollow expandable body interconnecting the end plates by one or more annular body section having radially spaced apart inner and outer side walls, the inner side wall defining therewithin a central channel axially extending between the central opening defined in each of the first and second end plates, the central openings permitting bone ingrowth therein and the central channel permitting bone growth fully therethrough between the two opposed end plates; and
wherein the hollow expandable body is axially expandable such as to extend from a collapsed position to an expanded position, the expanded position filling a space left by the at least one excised vertebral body.

2. The expandable vertebral prosthesis as defined in claim 1, further comprising an air evacuation system for evacuating air from out of the annular cavity, the air evacuation system including microscopic holes defined in at least one of the internal and external side walls of the body, the microscopic holes permitting air evacuation therethrough while preventing more viscous hardenable fluid to flow therethrough.

3. The expandable vertebral prosthesis as defined in claim 1, wherein the inner and outer side walls being concertinaed such that the annular body section forms a bellows.

4. The expandable vertebral prosthesis as defined in claim 3, wherein a sealed annular cavity is defined within the annular body section between the inner and outer side walls for receiving and retaining a hardenable material therein, the expandable body being axially expandable when said annular cavity is filled with the hardenable material.

5. The expandable vertebral prosthesis as defined in claim 1, wherein the hollow expandable body includes more than two concentric barrels interconnected and relatively displaceable such as to form a telescopic central body of the vertebral prosthesis.

6. The expandable vertebral prosthesis as defined in claim 5, wherein the telescopic central body is expandable between the collapsed position and the expanded position by a mechanical expansion mechanism interconnecting the multiple concentric barrels and operable to expand the telescopic central body into the expanded position absent any hardenable material.

7. The expandable vertebral prosthesis as defined in claim 6, wherein the expansion mechanism includes at least one of a screw, turnbuckle, ratchet, and a scissor-jack mechanism.

8. The expandable vertebral prosthesis as defined in claim 5, wherein the more than two concentric barrels of the telescopic central body comprise a plurality of nested barrels having different diameters.

9. The expandable vertebral prosthesis as defined in claim 8, wherein a sealed annular cavity is defined between the inner and outer side walls thereof for receiving and retaining a hardenable material therein, the telescopic central body being axially expandable from the collapsed position to the expanded position when said annular cavity is filled with the hardenable material.

10. The expandable vertebral prosthesis as defined in claim 1, wherein the opening in the opposed end pates has a bone growth stimulating material retained therewithin, the bone growth stimulating material substantially filling the central channel between the openings in the end plates when the body is in the collapsed position for insertion of the vertebral prosthesis.

11. The expandable vertebral prosthesis as defined in claim 1, further comprising stabilizing ties which position the inner side wall substantially centrally within the outer side wall, such as to maintain the channel centrally within the hollow expandable body of the vertebral prosthesis as the body expands from the collapsed position to the expanded position.

12. The expandable vertebral prosthesis as defined in claim 11, wherein said stabilizing ties are disposed within the annular cavity and extend substantially radially between the internal side wall and the external side wall.

13. The expandable vertebral prosthesis as defined in claim 1, wherein the vertebral prosthesis has an expansion ratio defined by a total axial height of the vertebral prosthesis in the expanded position divided by a total axial height of the vertebral prosthesis in the collapsed position, the expansion ratio being between about 20 percent and about 2000 percent.

14. The expandable vertebral prosthesis as defined in claim 13, wherein the expansion ratio is between about 300 and about 850 percent.

15. The expandable vertebral prosthesis as defined in claim 1, wherein a combined axial height of said end plates is less than 10 percent of the total axial height of the vertebral prosthesis when disposed in said collapsed position, said tubular side wall having an axial height of at least 90 percent of the total height of the vertebral prosthesis in said collapsed position.

16. The expandable vertebral prosthesis as defined in claim 15, wherein the combined axial height of said end plates is less than 5 percent of the total height of the vertebral prosthesis when disposed in said expanded position.

17. An expandable vertebral prosthesis comprising opposed first and second end plates spaced apart by a hollow expandable body and including outer surfaces thereon which are respectively adapted to abut adjacent vertebral bodies for engagement therewith, the end plates each having a bone ingrowth opening extending therethrough, the hollow expandable body having an annular configuration defining a central channel longitudinally extending therethrough between the bone ingrowth openings in the end plates, the central channel permitting bone growth fully therethrough between the opposed first and second end plates, the hollow expandable body having an external side wall and an internal side wall enclosing a sealed annular cavity therebetween, the internal side wall enclosing the central channel extending between said openings in the end plates, the annular cavity defined within the body being adapted to receive and contain a hardenable material therein, at least one inlet port being provided in fluid flow communication with said annular cavity to permit injection of the hardenable material into said cavity thereby expanding the body to expand the vertebral prosthesis in a longitudinal axial direction such that the end plates are displaced away from each other, the vertebral prosthesis being axially expandable when the annular cavity is filled with the hardenable fluid to extend the vertebral prosthesis from a collapsed position to an expanded position in order to fill a space between said adjacent vertebral bodies.

18. The expandable vertebral prosthesis as defined in claim 17, further comprising an air evacuation system for evacuating air from out of the annular cavity within said hollow expandable body, the air evacuation system including microscopic holes defined in at least one of the internal and external side walls of the body, the microscopic holes permitting air evacuation therethrough while preventing the more viscous hardenable fluid to flow therethrough.

19. The expandable vertebral prosthesis as defined in claim 17, wherein the opening in each of the first and second end pates has a bone growth stimulating material retained therewithin.

20. The expandable vertebral prosthesis as defined in claim 17, wherein the internal and external side walls are concertinaed such that the body defines an annular bellows, the central channel extending though the annular bellows between the openings in each of the end plates.

21. The expandable vertebral prosthesis as defined in claim 21, wherein a number of stabilizing ties extend substantially radially between the internal side wall and the external side wall of the body within the annular cavity, the stabilizing ties centrally locate the internal side wall and therefore position the central channel enclosed by the internal side wall,

22. The expandable vertebral prosthesis as defined in claim 17, wherein the vertebral prosthesis has an expansion ratio defined by a total axial height of the vertebral prosthesis in the expanded position divided by a total axial height of the vertebral prosthesis in the collapsed position, the expansion ratio being between about 20 percent and about 2000 percent.

23. A expandable vertebral prosthesis comprising opposed first and second end plates spaced apart by a hollow expandable body and including outer surfaces thereon which are respectively adapted to abut adjacent vertebral bodies for engagement therewith, the end plates each having a bone ingrowth opening extending therethrough, the hollow expandable body having an annular configuration defining a central channel longitudinally extending therethrough between the bone ingrowth openings in the end plates, the hollow expandable body having an external side wall enclosing the central channel extending between said openings in the end plates, the hollow expandable body including three or more concentric barrels nested together and relatively displaceable such as to form a telescopic central body of the vertebral prosthesis, the telescopic central body being extendable to expand the vertebral prosthesis in a longitudinal axial direction such that the end plates are displaced away from each other, the telescopic central body extending a determined distance to expand the vertebral prosthesis from a collapsed position to an expanded position in order to fill a space between said adjacent vertebral bodies.

24. The expandable vertebral prosthesis as defined in claim 23, wherein a mechanical expansion mechanism interconnects the concentric barrels and is operable to expand the telescopic central body a desired axial distance to expand the vertebral prosthesis into the expanded position absent any hardenable material.

25. The expandable vertebral prosthesis as defined in claim 24, wherein the expansion mechanism is disposed within the concentric barrels and includes at least one of a screw, turnbuckle, ratchet, and a scissor-jack mechanism.

26. The expandable vertebral prosthesis as defined in claim 23, wherein the concentric barrels of the telescopic central body define a sealed annular cavity between the inner and outer side walls thereof for receiving and retaining a hardenable material therein, the telescopic central body being axially expandable from the collapsed position to the expanded position when said annular cavity is filled with the hardenable material.
